(19) United States
(12) Patent Application Publication
(10) Pub. No.: US 2011/0144375 A1
(43) Pub. Date: Jun. 16, 2011

(54) PROCESS FOR PRODUCTION OF BIODIESEL

(76) Inventors: Rajiv Kumar Chaturvedi, Pune (IN); Nawalkishor Mal, Pune (IN); Kyatanahalli Srinivasa Nagabhushana, Pune (IN); Tushar R. Shinde, Pune (IN)

(21) Appl. No.: 12/996,088

(22) PCT Filed: May 21, 2009
(86) PCT No.: PCT/IN2009/000295

§ 371 Date: Feb. 23, 2011

(30) Foreign Application Priority Data
Jun. 4, 2008 (IN) 1181/MUM/2008

(51) Int. Cl. C07C 69/00

(52) U.S. Cl. 560/129

(57) ABSTRACT

A process for producing alkyl esters is disclosed. The process includes reacting a feedstock that includes one or more fatty acid glycerol esters or one or more fatty acids or mixture thereof with a C₁ to C₄ alcohol in the presence of fly ash as a catalyst. A catalyst for the production of alkyl esters is also disclosed.

The following disclosure generally relates to a process for production of alkyl esters. More particularly, the disclosure relates to a process for production of fatty acid alkyl esters useful in biofuels by esterification and/or transesterification reaction using fly ash as a catalyst.

### DESCRIPTION OF RELATED ART

Biodiesel is a non-petroleum based fuel that consists of fatty acid alkyl esters, made from vegetable oils or animal fats. The lower alkyl (C₁-C₄) esters generated from the oils and fats can be appropriately blended with petroleum diesel that makes the blend suitable for use in diesel engine. As biodiesel is a biodegradable and non toxic alternative to diesel fuel, it is becoming increasingly useful as a “green fuel”.

Commercial production of biodiesel is carried out by a transesterification reaction of vegetable oil or animal fat (hereafter called feedstock). Vegetable oil or animal fats is reacted with alcohol (e.g., methanol, ethanol) to convert the triglyceride in oils and fats to alkyl esters (biodiesel) and glycerine by-product. Since this reaction is slow, the reaction is carried out at elevated temperature and in the presence of a catalyst.

Most commercial processes for the production of biodiesel currently use a homogeneous alkali catalyst at 60-65° C. While the homogeneity of the reaction mass enhances the conversion rate, the catalyst is part of the reaction product. This makes it necessary to carry out a complicated step of separation and/or removal of the catalyst. The process of separating biodiesel from catalyst and glycerol involves a neutralization process with strong acids (e.g., HCl), and extensive washes with water to remove the resulting sodium salt. Further, in order to remove sodium chloride from glycerol and to obtain glycerol in high purity, distillation of high boiling glycerol has to be carried out which is an energy intensive operation.

The use of alkali catalyst also cause saponification of free fatty acids contained in fats and oils to form soaps as by products, whereby it becomes necessary to carry out a step of washing with large amounts of water. In addition, the yield of alkly esters (biodiesel) decreases due to the emulsification effect of the soaps generated and, in certain instances, the subsequent glycerine purification process also becomes complicated. In order to overcome the problem associated with free fatty acids, a strong homogeneous acid like sulphuric acid is generally used along with the reactant alcohol (e.g., methanol) as a pre-treatment catalyst that converts free fatty acids to alkyl esters. However, if acid is used in the pre-treatment process, neutralization of oil has to done before transesterification reaction may be carried out. This further creates economical and environmental concerns.

In order to overcome the problems associated with use of a homogeneous catalyst, heterogenous solid catalysts for the transesterification of oils to biodiesel have been developed. For example, various basic metal oxides, such as magnesium methoxide, calcium oxide, calcium alkoxide, and barium hydroxide, have been demonstrated to be active catalysts for transesterification.

However, the recyclability of these solid base catalysts is poor. This is because of the moderate solubility of some of these solid metal oxides, hydroxides and alcoxides in methanol/ethanol and strong physical adsorption of the reaction products on their surfaces.

Use of double metal cyanides and metal (e.g., Zn, Mo) embedded on supports (like alumina) as recyclable solid catalysts have also been claimed recently. The major drawback of such a catalyst is its relatively higher cost of preparation and therefore requiring large number of recycles. These recovery and further activation for recycling of catalyst cause technical and economic restrains.

In view of these drawbacks, there is a need to develop a process for biodiesel production that does not require tedious aqueous washes and neutralization steps. An economical and recyclable catalyst that can be easily separated from the biodiesel products for the conversion of oils to biodiesel is also needed. Moreover a catalyst that can economically catalyse both the esterification of free fatty acids and transesterify oils to biodiesel is desirable.

### SUMMARY

In one aspect a process for producing alkyl esters is disclosed. The process includes reacting a feedstock that includes one or more fatty acids, fatty acid glycerol esters or mixture thereof with a C₁ to C₄ alcohol in the presence of fly ash as a catalyst. The fly ash functions as a catalyst in the transesterification of the fatty acid glycerol esters and the esterification of fatty acid.

In another aspect a catalyst composite material for the production of alkyl esters from a feedstock including one or more fatty acid glycerol esters or one or more fatty acids or mixture thereof is disclosed. The catalyst composite material includes fly ash.

### BRIEF DESCRIPTION OF ACCOMPANYING FIGURES

The accompanying drawing illustrates the preferred embodiments of the invention and together with the following detailed description serves to explain the principles of the invention.

FIG. 1 illustrates X-Ray Diffraction spectrum of a sample of fresh and re-used fly ash catalyst.

### DETAILED DESCRIPTION

To promote an understanding of the principles of the invention, reference will be made to the embodiment and specific language will be used to describe the same. It will nevertheless be understood that no limitation of scope of the invention is thereby intended, such alterations and further modifications in the illustrated process and such further applications of the principles of the inventions as illustrated therein being contemplated as would normally occur to one skilled in art to which the invention relates.

A method for the production of alkyl esters is described. More particularly, a method of production of alkyl esters from a fatty acid containing feedstock using fly ash as a catalyst is described. Fly ash is generally defined as finely divided residue resulting from the combustion of powdered coal transported from the firebox through the boiler by the flue gases. The composition of the fly-ash is found to vary on the type of coal used. However, the composition of the fly ash typically has the following composition by weight percentage (5-12) SiO₂:(2-11) Al₂O₃:(0.50-2.0) Fe₂O₃:(35-60) CaO:(0.40-1) MgO:(26-30) SO₃.

The process for production of alkyl esters comprises reacting a feedstock that includes one or more fatty acid glycerol esters or one or more fatty acids or mixture thereof with an alcohol in the presence of fly ash as a catalyst to get a reaction mixture comprising a mixture of alkyl esters, alcohol and fly ash and recovering alkyl esters from the reaction mixture.

Fly ash used in the process catalyzes transesterification of the fatty acid glycerol esters present in the feedstock as illustrated in the exemplified reaction below:

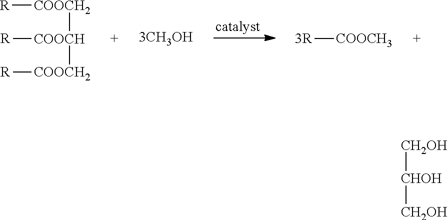

The fly ash also catalyzes the esterification of fatty acids present in the feedstock as illustrated in the exemplified reaction

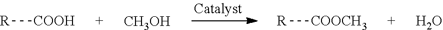

1-30 weight percentage of fly ash with respect to the fatty acid starting material may be used as a catalyst for the reaction. The molar ratio of the feedstock to alcohol may be in the range of 3 to 60, preferably in the range of 6 to 30 and most preferably in the range of 10 to 15.

A greater than 98% conversion is achieved by the process and a greater than 99% conversion is achieved using fly ash as a catalyst under preferred reaction condition.

Fly ash is easily recovered from the reaction mixture by any method including gravitational settling, filtration, centrifugation or any combination thereof.

In accordance with an aspect, once separated, the fly ash may be reused, if needed, as a catalyst for biodiesel production without any loss of catalytic activity. The recyclability has been tested for at least five cycles and the reaction proceeds with quantitative yield of the products.

In accordance with an aspect, the fly ash recovered from the reaction mixture may be washed and dried prior to reusing it as a catalyst for the production of alkyl esters. The fly ash recovered from the reaction mixture may be washed with any organic solvent in which the organics are soluble. In accordance with an aspect, hydroxylated solvents for example alcohols such as methanol and ethanol are used but less polar organic solvents like hydrocarbons (e.g., hexane) may also be used to selectively remove the biodiesel. Glycerine left behind with the catalyst may be extracted with water or a hydroxylated solvent. Chlorinated solvents such as chloroform, dichloromethane may also be used.

The reaction mixture includes an upper layer containing fatty acid alkyl esters and alcohol and a lower layer containing glycerol and alcohol. Recovery of alkyl esters from the reaction mixture is carried out by separating the catalyst from the reaction mixture. The alkyl esters are recovered from the upper layer and separated from the glycerol rich lower layer and alcohol is removed from the two layers. Alternatively, methanol can be distilled off by simply de-pressuring the reactor at the reaction temperature leaving behind two immiscible liquids in fatty acid alkyl ester and glycerol along with the solid catalyst.

In accordance with an aspect, the production of alkyl esters comprises of reacting a feedstock that includes one or more fatty acid glycerol esters or one or more fatty acids or mixture thereof with an alcohol in the presence of fly ash as a catalyst at an elevated temperature and autogenerated pressure for a predetermined period of time to get a reaction mixture, the reaction mixture contains a mixture of alkyl esters, glycerol, alcohol and fly ash; removing the fly ash catalyst from the said reaction mixture by filtration or any suitable conventional separation method to get a liquid with two phases, an alcohol containing alkyl esters rich upper layer and alcohol containing glycerol rich lower layer, separating the two phases and removing the alcohol from alkyl esters and glycerol rich liquids by conventional distillation to get alkyl esters and glycerol.

The feed stock used for this process may contain free fatty acids or fatty acid glycerol esters or their mixture thereof. The fatty acid glycerol esters may be mono-, di- or tri-ester of glycerol with varying degree of unsaturation in the fatty acid chain. The feedstock used for the production of alkyl esters may be any fatty acid rich material including but not limited to vegetable oil, used vegetable oil, restaurant waste grease, acid oil or surplus liquid or solid fats such as vegetable shortening, surplus margarine or animal fats. Each of these may be used individually or as a mixture.

In accordance with an aspect, additional processing such as removal of excess water or filtering out of precipitate may be required before using animal fat or vegetable oil for this process.

The alcohol to be used for the reaction may be any C₁ to C₄ alcohol, including but not limited to methanol, ethanol, propa(en)nol and buta(en)nol. The alcohol used can be primary, secondary or tertiary in nature. Single alcohol or a mixture of two or more alcohols may also be used for the reaction.

In accordance with an aspect, the reaction is carried out at an elevated temperature of 120-250° C. under autogenerated pressures.

In accordance with an aspect, the alcohol containing alkyl ester rich upper layer may be separated from the alcohol containing glycerol rich lower layer by any method including but not limited to gravitational settling, centrifugation, distillation, using separation funnel or a combination thereof. In accordance with an embodiment alcohol is removed from alkyl esters and glycerol by vacuum distillation.

A catalyst composite material for the production of alkyl esters useful in biofuels from a feedstock including one or more fatty acid glycerol esters and one or more fatty acids, is also disclosed. The catalyst composite material includes fly ash. In accordance with an aspect the catalyst composite material includes at least 1 weight percentage of fly ash. In accordance with an aspect the catalyst composite material includes 30 weight percentage of fly ash.

Specific Embodiments are Described Below:

A process for production of alkyl esters comprises reacting a feedstock that includes one or more fatty acid glycerol esters or one or more fatty acids or mixture thereof with a C1 to C4 alcohol in the presence of fly ash as a catalyst.

A process, wherein the reaction is carried out at a temperature between 120° C. and 250° C. under autogeneous pressure for a period of 30 minutes to 8 hours.

A process, wherein the fly ash functions as a catalyst in the transesterification of the fatty acid glycerol esters and the esterification of fatty acids.

A process, wherein the amount of fly ash used as a catalyst is in the range of 1 to 30 weight percentage with respect to the feedstock.

A process, wherein the fly ash comprises of two or more of SiO₂, Al₂O₃, Fe₂O₃, CaO, MgO and SO₃.

A process, wherein the fly ash comprises of 5 to 12 weight percentage SiO2:2 to 11 weight percentage Al2O3:0.50 to 2.0 weight percentage Fe2O3:35 to 60 weight percentage CaO:0.40 to 1 weight percentage MgO:26 to 30 weight percentage SO3.

A process, wherein the process further comprises of recovering the fly ash from the reaction mixture.

A process, wherein the recovered fly ash is recycled.

A process, wherein the process further comprises of separating the fly ash from the reaction mixture; washing and drying the washed fly ash and reusing the fly ash as a catalyst for producing alkyl esters.

A process, wherein the fatty acid ester is a mono-, di- or tri-ester of glycerol with varied degree of unsaturation in the fatty acid chain.

A process, wherein the alcohol is any of methanol, ethanol, propan(en)ol or butan(en)ol or their mixtures.

A process, wherein the molar ratio of the feedstock to alcohol can be in the range of 3 to 60 preferably in the range of 6 to 30 and most preferably in the range of 10 to 15.

A catalyst composite material for the production of alkyl esters from a feedstock including one or more fatty acid glycerol esters or one or more fatty acids or their mixture, wherein the catalyst includes fly ash.

A catalyst composite material, wherein the amount of fly ash in the catalyst composite material is at least 30 weight percentage and the remaining may be any inert or active component in the catalysts composite material.

A catalyst composite material including fly ash wherein the fly ash comprises of 5 to 12 weight percentage SiO2:2 to 11 weight percentage Al2O3:0.50 to 2.0 weight percentage Fe2O3:35 to 60 weight percentage CaO:0.40 to 1 weight percentage MgO:26 to 30 weight percentage SO3.

A catalyst composite material wherein the fly ash comprises of two or more of SiO₂, Al₂O₃, Fe₂O₃, CaO, MgO and SO₃.

The following examples are provided to explain and illustrate certain preferred embodiments of the process of the invention.

### Example 1

300 g of soybean oil, 150 g of methanol and 30 g of fly ash were put in batch reactor maintained at different reaction temperatures in the range of 120 to 230° C. under auto generated pressure for 6 h after attaining the set temperature. At the end of this time, the reaction mass was allowed to reach room temperature, product mixture drained and catalyst filtered off. Two layers of liquid were present, upper one containing diesel in methanol and glycerol is present in lower layer along with methanol. Two layers of liquid were separated using separation funnel. Methanol was removed by distillation from both the layers separately. Similarly, methanol was distilled off by just de-pressurising the reactor and allowing the reminders of the reactor to settle down into two distinct layers and separating the solid catalyst by filtration. In either case conversions as high as >98% could be achieved using fly ash as catalyst. The products of the reactions were analyzed by the standard ASTM D 6584 method (Gas Chromatography method) protocols using silylating agent to derivatize the reaction components to be quantified. Reproducibility of results and comparison of the percentage conversions were easily and routinely performed. The vegetable oil conversion in to biodiesel at 120° C. and 150° C. was 70% and 85% respectively. However at above 170° C., complete feed stock conversion (99%+) could be achieved.

### Example 2

300 g of pongamia oil, 150 g of methanol and 30 g of fly ash were put in batch reactor maintained at 180° C. under auto generated pressure for 3 h. At the end of this time, the reaction mass was allowed to reach room temperature, product mixture drained and catalyst filtered off. Two layers of liquid were present, upper one containing diesel in methanol and glycerol is present in lower layer along with methanol. Two layers of liquid were separated using a separation funnel. Methanol was removed by vacuum distillation from both the layers separately. More than 98% conversion into biodiesel was achieved with complete utilization of the triglycerides. However, the biodiesel obtained by this method had coloration since the starting material was also highly coloured.

### Example 3

300 g of pongamia oil, 150 g of methanol and 30 g of fly ash were put in batch reactor maintained at 180° C. under auto generated pressure for 3 h. At the end of this time, the reaction mass was allowed to reach room temperature, product mixture drained and catalyst filtered off. The separation was performed as described in example 1. Complete conversion of the triglycerides were seen with more than >98% yield of the biodiesel. This indicates the general applicability of fly ash as a versatile cheap, reusable transesterification catalyst to convert triglycerides into biodiesel.

### Example 4

300 g of feed stock (containing 50 weight percentage of free fatty acid and 50 weight percentage soybean oil), 150 g of methanol and 30 g of fly ash were put in a batch reactor maintained at 180° C. under auto generated pressure for 3 h. At the end of this time, the excess methanol was distilled off by simply depressurising, as explained in example 1 and the two layers of liquid present in the reactor were separated. More than 95% yields of fatty acid methyl ester (FAME) and quantitative yields of glycerol were obtained.

### Example 5

300 g of pongamia oil, 150 g of methanol and 30 g of fly ash were simultaneously put in batch reactor maintained at 180° C. under auto generated pressure. After every 30 minutes from the start of the reaction that is after attaining the set temperature, a small portion of the reaction mass was withdrawn and analyzed by Gas Chromatography method using standard ASTM protocols as described before. At the end of two hours, the reaction mass was allowed to reach room temperature, product mixture drained and catalyst filtered off. Two layers of liquid were present, upper one containing bio-diesel in methanol and glycerol is present in lower layer long with methanol. Two layers of liquid were separated using separation funnel. Table 1 depicts the rate of completion of the reaction using 10 weight percentage of the catalyst.

TABLE 1
Percentage conversion of the triglyceride with time
using 10 weight percentage of fly ash as catalyst.

| S. No. | Time in Minutes | % conversion of triglyceride |
|---|---|---|
| 1 | 30 | 80 |
| 2 | 60 | 90 |
| 3 | 90 | 95 |
| 4 | 120 | 98 |
| 5 | 150 | >99 |
| 6 | 180 | >99 |

### Example 6

300 g of pongamia oil, 150 g of methanol and various amounts of fly ash (1-15 weight percentage) were put in batch reactor maintained at 180° C. under auto generated pressure for different time periods. At the end of this time, the reaction mass separated into individual fractions and percentage conversions are recorded as shown in Table 2. Although, still higher amount of catalyst (say 20% and 30%) can easily be used but this not preferred as it may pose problems in down steam processing.

TABLE 2
Transesterification of pongamia oil
using different amount of catalyst.

| S. No. | Weight percentage of catalyst | % conversion |
|---|---|---|
| 1 | 1 | 90 |
| 2 | 3 | 95 |
| 3 | 5 | 98 |
| 4 | 7.5 | 99 |
| 5 | 10 | >99 |
| 6 | 15 | >99 |

### Example 7

This example illustrates the effect of methanol to soybean oil molar ratio, which was varied between 4.5 to 45. While at a methanol to vegetable oil molar ratio of 7.5 and above, almost complete conversion of feed stock was obtained in about 2 to 4 hours at 180° C. At a lower methanol content (methanol to feedstock molar ratio equal or less that 4.5), about 90% feedstock conversion to biodiesel was observed.

Alcohols including ethanol, propanol and butanol were all tried and they resulted in the formation of fatty acid methyl ester (FAME) with the corresponding alcohol components in them in quantitative yields when the alcohol to oil molar ratio was 15:1.

### Example 8

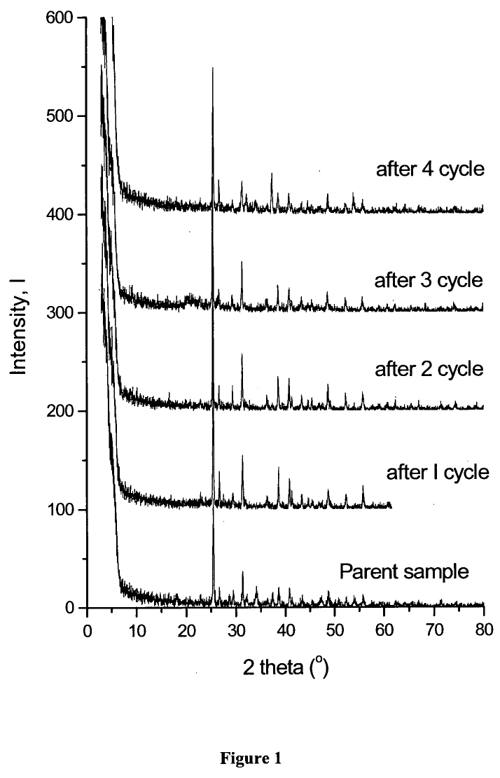

Experiment as depicted in example 1 was repeated with the re-cycled catalyst. The catalytic reactions were repeated for Soybean oil for more than 5 times and no reduction in the catalytic activity was seen indicating no or very small leaching of the metal into the reaction mass. This was also evident from the X-Ray Diffraction spectrum of a sample of the fresh and re-used fly ash catalyst as illustrated in FIG. 1.

### INDUSTRIAL APPLICABILITY

The process as described produces biodiesel in an economically efficient and an environmental friendly manner. As fly ash is a solid catalyst, it can be easily separated from the reaction mixture and re-used thereby eliminating the need of neutralization step and aqueous washes that are associated with use of conventional catalysts. Moreover, as fly ash catalyses both the esterification reaction of the free fatty acids and the transesterification of triglycerides that are present in the fatty acid starting material (free fatty acids and, oils and fats). The process has several advantages. Firstly, the efficiency of the process increases since no acid pre-treatment process and subsequent neutralization steps are needed. Also, biodiesel along with glycerine is generated as the only reaction product without any contaminations. This enables easy separation of the two immiscible layers from the catalyst, yielding biodiesel in quantitative yield that needs no further purification. The contaminations can only come from such sources where the free acid contents are higher than 20 weight percentage in the oil, as seen in the case of acid oil. Fly ash separated from the reaction mixture does not lose its catalytic activity and may be reused as a catalyst, thereby reducing the cost of biodiesel production. Moreover, the use of fly ash provides an alternate to the disposal related concerns of fly ash generated in industries.

The embodiments of the invention, described above, are intended to be exemplary, and not limiting. Many variations are possible, within the scope of the invention. These and other modifications are to be deemed within the spirit and scope of the following claims.

1. A process for production of alkyl esters comprises reacting a feedstock that includes one or more fatty acid glycerol esters or one or more fatty acids or mixture thereof with a C₁ to C₄ alcohol in the presence of only fly ash as a catalyst at a temperature between 120° C. and 250° C., wherein the fly ash functions as a catalyst in the transesterification of the fatty acid glycerol esters and/or the esterification of fatty acids.

2. A process as claimed in claim 1, wherein the reaction is carried out under autogeneous pressure for a period of 30 minutes to 8 hours.

3. (canceled)

4. A process as claimed in claim 1, wherein the amount of fly ash used as a catalyst is in the range of 1 to 30 weight percentage with respect to the feedstock.

5. A process as claimed in claim 1, wherein the fly ash comprises of two or more of SiO₂, Al₂O₃, Fe₂O₃, CaO, MgO and SO₃.

6. A process as claimed in claim 1, wherein the fly ash comprises of 5 to 12 weight percentage SiO₂:2 to 11 weight percentage Al₂O₃:0.50 to 2.0 weight percentage Fe₂O₃:35 to 60 weight percentage CaO:0.40 to 1 weight percentage MgO:26 to 30 weight percentage SO₃.

7. A process as claimed in claim 1, wherein the process further comprises of recovering the fly ash from the reaction mixture.

8. (canceled)

9. A process as claimed in claim 6, wherein the process further comprises of separating the fly ash from the reaction mixture; washing and drying the washed fly ash and reusing the fly ash as a catalyst for producing alkyl esters.

10. A process as claimed in claim 1, wherein the fatty acid ester is a mono-, di- or tri-ester of glycerol with varied degree of unsaturation in the fatty acid chain.

11. A process as claimed in claim 1, wherein the alcohol is any of methanol, ethanol, propan(en)ol or butan(en)ol or their mixtures.

12. A process as claimed in claim 1, wherein the molar ratio of the feedstock to alcohol can be in the range of 3 to 60 preferably in the range of 6 to 30 and most preferably in the range of 10 to 15.

13. Alkyl esters obtained by a process as claimed in any preceding claim.

14.-17. (canceled)

18. A process for producing alkyl esters substantially herein described with reference to and as illustrated by the accompanying figure.

19. (canceled)

20. A process as claimed in claim 5, wherein the fly ash comprises of 5 to 12 weight percentage SiO₂:2 to 11 weight percentage Al₂O₃:0.50 to 2.0 weight percentage Fe₂O₃:35 to 60 weight percentage CaO:0.40 to 1 weight percentage MgO:26 to 30 weight percentage SO₃.
